(19) United States
(12) Patent Application Publication
(10) Pub. No.: US 2010/0318091 A1
(43) Pub. Date: Dec. 16, 2010

(54) PLASTICIZED MATERIAL, DELIVERY DEVICE AND METHOD FOR FILLING A BONE CAVITY AND INCLUDING BOTH FOAM PLASTIC SPRAY AND INJECTED LIQUID PELLETS AND FOR PROMOTING BONE GROWTH AND ADHESION

(75) Inventor: Miguel A. Linares, BLOOMFIELD HILLS, MI (US)

Correspondence Address: GIFFORD, KRASS, SPRINKLE,ANDERSON & CITKOWSKI, P.C, PO BOX 7021, TROY, MI, 48007-7021, US

(73) Assignee: LINARES MEDICAL DEVICES, LLC, Auburn Hills, MI (US)

(21) Appl. No.: 12/794,852

(22) Filed: Jun. 7, 2010

Related U.S. Application Data
(60) Provisional application No. 61/185,823, filed on Jun. 10, 2009.

(51) Int. Cl. A61B 17/58; A61F 2/28

(52) U.S. Cl. 606/94; 523/116; 521/50

(57) ABSTRACT

An artificial material, kit and process for filling a three dimensional cavity associated with a damaged or diseased bone and including a body composed of a cellular material including either a foam plastic spray or a syringe holding a plurality of liquid pellets and filling a preconditioned area associated with the bone. The syringe includes a stem which supports, at an extending end, a flexible forming guide for covering an inside location associated with the cavity concurrent with filling the interior. The material sets to facilitate long term and rehabilitative bone growth within and through the cells.

### CROSS-REFERENCE TO RELATED APPLICATIONS

This application claims the benefit of U.S. Provisional Application 61/185,823 filed on Jun. 10, 2009.

### FIELD OF THE INVENTION

The present invention discloses an artificial material, delivery device and process for filling a three dimensional cavity associated with such as a damaged or diseased bone. More specifically, the present invention discloses a foam plastic spray as well as a liquid pellet application, such as utilizing a delivery mechanism device for filling a preconditioned and hollowed area associated with a bone and for facilitating long term and rehabilitative bone growth.

### BACKGROUND OF THE INVENTION

The relevant background art is directed to the incidence of cavities or depressions within bone structure, this resulting from either injury or disease. It is desirous to effectively seal or fill a bone cavity, such as in situ within the patient.

### SUMMARY OF THE INVENTION

The present invention discloses an artificial material and associated delivery mechanism for filling a three dimensional cavity associated with a damaged or diseased bone and including a body composed of a cellular material including either a foam plastic spray or a syringe holding a plurality of liquid pellets and filling a preconditioned area associated with the bone. The syringe includes a stem which supports, at an extending end, a flexible forming guide covering an inside location proximate a surface associated with the cavity concurrent with filling the interior. The material sets to facilitate long term and rehabilitative bone growth within and through the cells. An associated process for filling the bone cavity includes the insertion of the syringe with flexible end forming guide within the narrowed inlet defining aperture, the progressive in fill application of the resinous material/plastic pellets, and the subsequent fracture removal of the syringe stem so as to leave in place the forming guide in abutting contact with inner surfaces associated with the narrowed inlet.

### BRIEF DESCRIPTION OF THE DRAWINGS

Reference will now be made to the attached drawings, when read in combination with the following detailed description, wherein like reference numerals refer to like parts throughout the several views, and in which:

FIG. 1 is a representative illustration of a foam plastic spray according to a first variant and which is applied to a bone recess location for promoting long term bone growth and adhesion;

FIG. 2 is an enlarged illustration of a spray location associated with FIG. 1 and further showing the manner in which new bone growth is facilitated by the interspatial configuration of the individual foam plasticized cells;

FIG. 3A is an illustration of a bone patch according to a second preferred embodiment and in which a uniquely configured and combination injecting syringe with needle end located and surface forming guide is shown in a first pre-engaged position relative to a previously hollowed out and pre-conditioned location of an existing bone;

FIG. 3B is a succeeding illustration in which the surface located forming guide is pre-located in inner seating fashion associated with a narrowed inlet associated with the hollowed out and pre-conditioned bone;

FIG. 3C is a further succeeding illustration in which a volume of liquid pellets are injected through the syringe and into the three dimensional interior associated with the pre-conditioned bone aperture, the location of the surface forming guide preventing pellets from spilling out of the bone recess prior to solidifying and curing;

FIG. 3D is a yet further succeeding illustration in which the injected pellets solidify in a desired interstitially spaced fashion permissive for subsequent inter bone growth, the forming guide further capable of being snap-detached from an associated end of the syringe and subsequently functional as a permanent cover portion;

FIG. 4 is an enlarged illustration of the arrangement shown in FIG. 3D and better illustrating the liquid pellets contained within the syringe and deposited into the pre-conditioned bone aperture;

FIG. 5A is a first plan view illustration generally corresponding to the initial insertion of the syringe with forming guide (FIG. 3A) in a similar bone insertion application and in which the forming guide is initially located against an inner end surface of the pre-conditioned bone aperture prior to administering of the liquid pellets; and

FIG. 5B is a succeeding plan view of the syringe with forming guide generally corresponding to that shown in FIG. 3C in which the deposited volume of liquid pellets fills the volume associated with the bone aperture, concurrent with the guide establishing a sealing and spill-proof engagement with the inner seating location of the bone aperture inlet.

### DETAILED DESCRIPTION OF THE PREFERRED EMBODIMENTS

The present invention discloses a series of illustrations directed to embodiments particularly suited to the repair and regeneration of bone growth in a pre-conditioned area associated with a damaged or diseased bone. In particular, and as will be described in further detail with reference to the succeeding illustrations, the invention discloses, according to a pair of desired but non-limiting variants, the application of a synthetic formable material, including such as foam plastic and liquid pellet plastic variants, for filling and sealing such a preconditioned or medically prepared aperture location associated with a damaged bone, as well as for providing for future bone growth in intermixing fashion with the plastic and so that the fashioned three dimensional patch thus created functions as a permanent part of the bone thereafter.

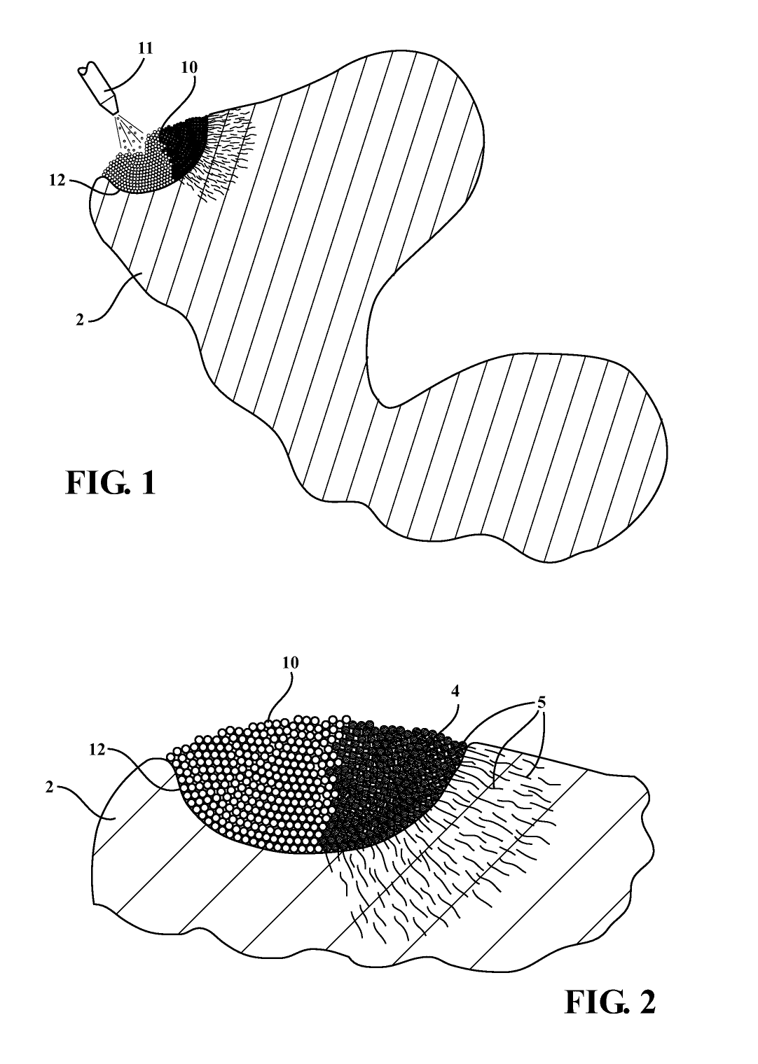

Referring to FIG. 1, a representative illustration is shown of a foam plastic spray composition 10 according to a first variant and which is applied via a delivery mechanism (see nozzle 11 illustrated in cutaway) to a recess location 12, such as of a skull 2, for promoting long term bone growth and adhesion. FIG. 2 is an enlarged illustration of the spray location associated with FIG. 1 and further showing the manner in which new bone growth, generally referenced at 4, is facilitated by the interspatial configuration of the individual foam plasticized cells associated with the spray composition 10.

The spray composition can include such as a micro-cellular plastic foam which is specially foamed so as to create micro-pores or cells. The common definition includes foams with pore size of varying diameter. In certain instances, foam cells of relatively small size retain the appearance and functionality of solid plastic.

Microcellular foams have also been constructed with density ranges of 5 to 99% of a base material. The microcellular foam plastics can be produced by any of injection molding, extrusion and blow molding processes, the advantages of which include a reduction of materials consumption, accuracy, long-term stability, higher productivity due to shorter cycle time, and the like.

Certain brands of microcellular (foam) plastics are created by a solid-state foaming process which saturates a thermoplastic with an inert gas at very high pressures. The gas dissolves in plastic, which absorbs the gas like a sponge. Subsequent heating of the polymer above an effective glass transition temperature (of the polymer/gas mixture) then causes the plastic to foam, creating a very uniform structure of small bubbles and can yield superior mechanical properties.

As best depicted in FIG. 2, and following deposition and hardening of the microcellular foam 10 within the pre-conditioned bone cavity, interstitial apertures 5 defined between the individual cells enables the ossification process of bone formation to occur and in which connective tissues, such as cartilage, are converted to bone or bone-like tissue. The ossified tissue is invaginated with blood vessels, which in turn bring minerals such as calcium and deposit it in the ossifying tissue. As is further known, such bone formation is a dynamic process continuing throughout the life of the individual, with cells called osteoblasts depositing minerals, and osteoclasts removing bone.

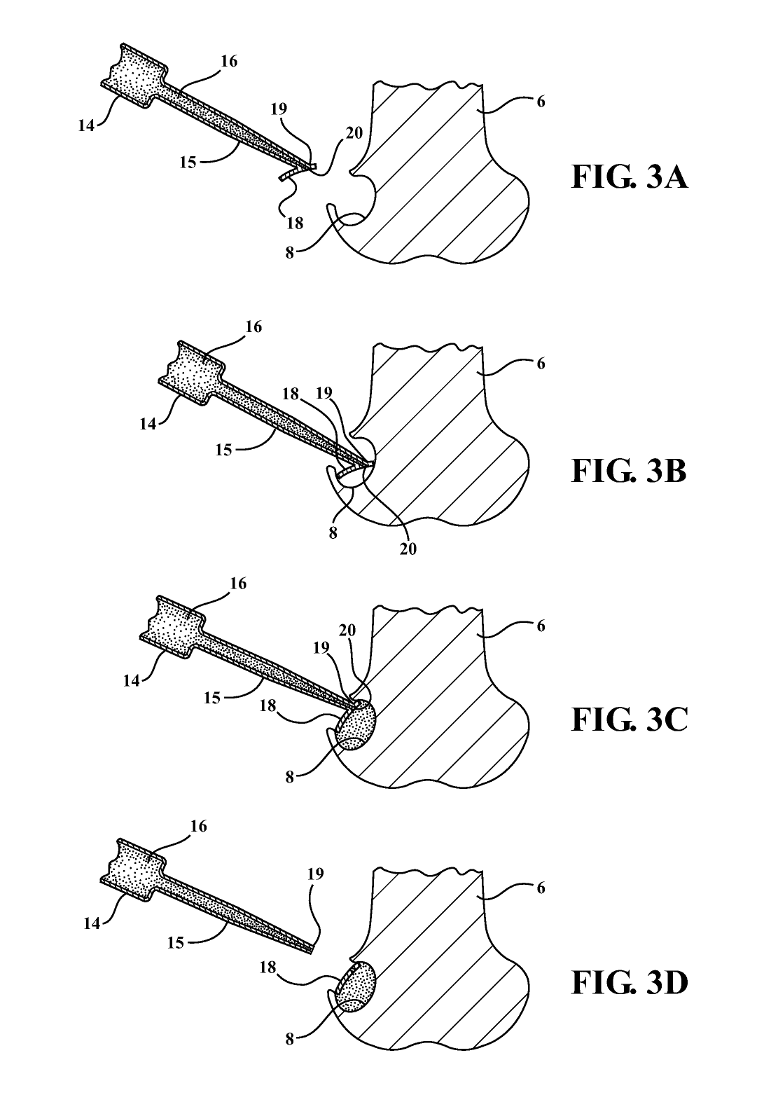

Referring now to FIG. 3A, an illustration is shown of a bone patch application according to a second preferred embodiment and in which a further variant of delivery mechanism, in the instance a uniquely configured and combination injecting syringe 14, is provided for injecting a filler material directly into a bone aperture 8, such as constituting a pre-conditioned or hollowed out area 8 corresponding to a previously damaged area which has been prepared for insertion of the patching material. The syringe 14 exhibits a generally barrel shaped end which, in combination with an extending stem 15, is loaded with a volume of liquid plastic filled and hardenable pellets 16.

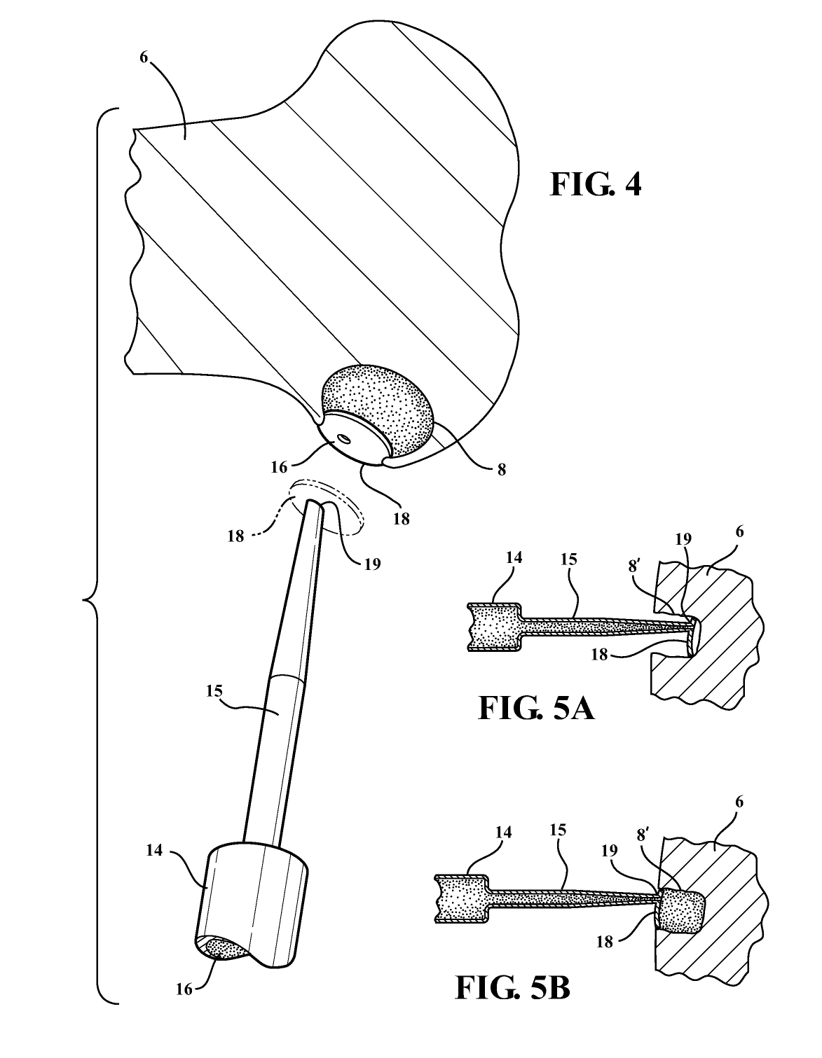

Further mounted to a communicating needle end of the stem portion 15 of the syringe 14 is located a generally planar shaped and flexible surface forming guide 18, this being shown in each of FIGS. 3A-3D in side cutaway profile as well as further shown in perspective with optional circular perimeter configuration in phantom in FIG. 4. The forming guide 18 can further exhibit any generally planar shape, including such as square or circular shapes, and is further constructed of a generally thin, flexible and, optionally, settable/hardenable material having a sufficient and form retaining thickness while also be permissibly flexible for deformably inserting within the pocket shaped aperture defined by the inner recesses surface 8 of the bone. The bone, as again representatively shown at 6 in each of FIGS. 3A-3D, 4 and 5, includes such a pre-conditioned, or interiorly hollowed out, portion as previously identified at 8 and such as which corresponds to an in-situ pre-repair operation performed upon a diseased or damaged area of the bone, which is desired to be reconditioned rather than requiring subsequent bone removal/replacement.

As again shown in FIG. 3A, the hollow stem portion of the syringe communicates an aperture 20 (see as best shown in FIGS. 3A and 3B) extending through the barrel, stem 15 to a communicating and interior location of the flexible and syringe end located forming guide 18, the forming guide 18 being mounted to the end of the stem 15 in order to be repositioned from a first pre-engaged position relative to the previously hollowed out and pre-conditioned location 8 of the existing bone 4 (again FIG. 3A) to the succeeding illustration of FIG. 3B in which the surface located forming guide 18 is pre-located in inner seating fashion beyond the narrowed inlet associated with the hollowed out and pre-conditioned bone aperture 8 and in proximity to an inner-most located portion of the interior volume defined by the aperture 8.

FIG. 3C is a further succeeding illustration in which a volume of the liquid pellets 16 (such as which can exhibit a desired cellular space and which may also include a fast drying/setting thermoform material into which a catalyst is introduced just prior to application) are injected through the syringe 14 and in order to fill the three dimensional interior associated with the pre-conditioned bone aperture 8. As is further shown, the location of the flexible surface forming guide 18 in seating fashion against the reduced dimension underside of the bone aperture prevents pellets from spilling out of the bone recess prior to solidifying and curing. It is also envisioned and understood that the pellets can exhibit any desired properties of thermal or chemical expansion, and so that a desired injected volume of pellets corresponds to an eventual three dimensional space occupied by the hardened/cured pellets taking into further account a desired degree of interstitial spacing established by the individual pellets for facilitating subsequent bone marrow growth within and through the patch matrix created by the pellets 16.

FIG. 3D is a yet further succeeding illustration in which the injected pellets 16 solidify in a desired interstitially spaced fashion permitting subsequent inter bone growth, such as in the fashion previously described in reference to FIG. 2. The pellets are fast drying and, at an appropriate point where they are semi-hardened, turning or twisting of the syringe 14 results in the forming guide 18 breaking off from the stem portion (due further to a weakened structural connection which is designed into the interface between the guide 18 and syringe stem), snap-detached guide subsequently functioning as a permanent cover or patch during subsequent hardening and ongoing use of the bone.

FIG. 4 is an enlarged illustration of the arrangement shown in FIG. 3D and better illustrating the liquid pellets 16 contained within the syringe 14 and deposited in a progressively filling fashion from an inner base surface associated with the pre-conditioned bone aperture 8. The flexible guide 18 (in phantom when attached to the end of the syringe) is also shown in permanently located and anchored fashion abutting against an inside entranceway of the interior formed pocket established by inner surface 8, and following being broken off from the terminating stem portion and following pre-insertion and filling of the bone aperture interior. In this application, a boundary location 19 established between the tip (or nozzle) end of the stem 15 and the flexible and covering/forming guide 18 consists of a frangible or breakable location (such as an intentionally weakened plastic connection) for permitting the stem 15 to be broken off as depicted in each of FIGS. 3D and 4.

Referring now to FIG. 5A, a first plan view illustration is shown of a similar bone aperture filling location, such as further identified by inner cavity defining surface 8′ arranged at a further location in comparison to that depicted in FIG. 3A and again generally corresponding to the initial insertion of the syringe with forming guide 18 (FIG. 3A), and in which the forming guide is again initially located proximate and against an inner end surface of the pre-conditioned bone aperture prior to administering of the liquid pellets 16. FIG. 5B is a succeeding plan view of the syringe with forming guide 18, also generally corresponding to that shown in the similar application step of FIG. 3C, and in which the deposited volume of liquid pellets 16 fills the interior volume associated with the bone aperture, this concurrent with the guide establishing a sealing and spill-proof engagement with the inner seating location of the bone aperture inlet.

Upon sufficient setting of the pellets 16, the stem 15 is pivoted and/or rotated to fracture remove from the base mounting location 19 of the covering guide 18, and which at this point is biased in an interiorly seated fashion against the inside narrowed profile of the bone aperture (see again as best shown in FIG. 3D), again following the hardening of the previously introduced pellets. The present invention also discloses associated methods for reconditioning the damaged/diseased bone, applying the micro-cellular foam plastic or liquid plastic pellets, manipulating the syringe with form shaping guide, and fracture removing the syringe stem following hardening/setting of the plastic pellets.

The associated delivery device and method of filling includes the features of reconditioning the bone cavity, inserting the delivery mechanism, including the spray nozzle or syringe with flexible end forming guide within the narrowed inlet defining aperture. In the instance of the spray nozzle the reconditioned bone cavity is filled with the micro-cellular material, and in the further example of the syringe progressive in filling of a resinous material in the form of plastic pellets. In the example of the syringe, additional steps include the subsequent fracture removal of the syringe stem so as to leave in place the forming guide in abutting contact with inner surfaces communicating with the narrowed inlet.

Having described my invention, other and additional preferred embodiments will become apparent to those skilled in the art to which it pertains, and without deviating from the scope of the appended claims.

I claim:

1. An artificial material for filling a three dimensional cavity associated with a damaged or diseased bone, comprising a body composed of a material filling an interior volume associated with the cavity, the material setting to facilitate long term and rehabilitative bone growth within and through said cells.

2. The invention as described in claim 1, said material further comprising a micro celluar foam plastic spray.

3. The invention as described in claim 1, said material further comprising a plurality of liquid pellets.

4. The invention as described in claim 3, further comprising a syringe filled with said liquid pellets.

5. The invention as described in claim 4, said syringe including a stem which supports, at an extending end, a flexible forming guide for covering an inside location associated with said cavity concurrent with filling said interior.

6. The invention as described in claim 5, further comprising a frangible breakaway location established between a nozzle end of said stem and said forming guide.

7. A device for filling a three dimensional cavity associated with a damaged or diseased bone, comprising a delivery mechanism for introducing a body composed of a synthetic formable material filling an interior volume associated with the cavity, the material setting to facilitate long term and rehabilitative bone growth within and through said cells.

8. The device as described in claim 7, said delivery mechanism further comprising a spray nozzle for introducing a micro cellular plastic foam.

9. The device as described in claim 7, said delivery mechanism further comprising a syringe with a flexible end forming guide for introducing a plurality of plasticized liquid pellets.

10. The device as described in claim 9, said syringe further comprising an elongated stem portion exhibiting an end defined fracture location to which said forming guide is connected in breakaway fashion.

11. A process for filling a bone cavity comprising the steps of:
inserting a nozzle portion of a delivery mechanism into an opening associated with the cavity; and
in filling an interior volume defined by the cavity with a formable material.

12. The process as described in claim 11, said step of in filling with a formable material further comprising admitting a micro-cellular material.

13. The process as described in claim 11, said step of in filling further comprising admitting a plurality of plastic liquid filled pellets.

14. The process as described in claim 13, further comprising the step of inserting into the bone cavity interior a flexible forming guide extending from said nozzle portion and prior to infilling with the pellets.

15. The process as described in claim 14, further comprising the steps of engaging edges of the forming guide against inner cavity surfaces associated with a narrowed inlet and fracture removing from the nozzle portion.
